(19) United States
(12) Patent Application Publication
(10) Pub. No.: US 2026/0083898 A1
(43) Pub. Date: Mar. 26, 2026

(54) CONNECTION DEVICE FOR CREATING A VACUUM-TIGHT FLUIDIC CONNECTION BETWEEN A VACUUM WOUND DRESSING AND A VACUUM SOURCE, VACUUM WOUND THERAPY KIT, AND VACUUM WOUND THERAPY SYSTEM

(71) Applicant: PAUL HARTMANN AG, Heidenheim (DE)

(72) Inventor: Martin DEIBLER, Herbrechtingen (DE)

(73) Assignee: PAUL HARTMANN AG, Heidenheim (DE)

(21) Appl. No.: 19/140,246

(22) PCT Filed: Dec. 13, 2023
(86) PCT No.: PCT/EP2023/085507

§ 371 Date: Jun. 17, 2025

(30) Foreign Application Priority Data
Dec. 19, 2022 (DE) 10 2022 133 928.9

(51) Int. Cl. A61M 1/00; A61F 13/05

(52) U.S. Cl. CPC A61M 1/912; A61F 13/05

(57) ABSTRACT

The invention relates to a connection device (22) for creating a vacuum-tight fluidic connection between a vacuum wound dressing (16) and a vacuum source (12), said device comprising: an elongated single-or multi-lumen connecting tube (24), in particular a film tube, which comprises a distal end portion (28) and a proximal end portion (26), it being possible to fluidically connect the distal end portion to the vacuum wound dressing in a vacuum-tight manner, and it being possible to fluidically connect the proximal end portion to the vacuum source in a vacuum-tight manner; and a support unit (46) which is located in a tube lumen (38) of the connecting tube, the support unit preventing the connecting tube from collapsing, in particular collapsing due to vacuum conditions, and being permeable to fluid in the longitudinal extension of the tube lumen. According to the invention: the support unit comprises at least one flat material web portion (48); the flat material web portion, in order to support the connecting tube, is structured by elevations (54) formed integrally with a plane (52) of the flat material web portion; and a continuous intermediate space (56) is formed between the elevations so that fluid can be transported through the intermediate space in the longitudinal extension of the tube lumen.

The present invention relates to a connecting device for the negative pressure-tight fluidic connection of a negative pressure wound dressing to a negative pressure source. The present invention moreover relates to a negative pressure wound therapy kit having such a connecting device. The present invention furthermore relates to a negative pressure wound therapy system comprising a negative pressure source and such a wound therapy kit.

Negative pressure wound therapy, or NPWT, is an innovative method for wound treatment with a wide range of indications. These include for example acute skin or soft tissue defects, wound healing problems, chronic wounds, etc. The aim of negative pressure therapy is to stimulate the growth of granulation tissue and to promote the healing process. In the scope of negative pressure therapy, for this purpose a negative pressure is established in the region of a wound so that wound exudate can be removed effectively from the wound.

Negative pressure wound dressings used in negative pressure therapy typically comprise an air-impermeable cover layer for airtight sealing of the wound. An attachment opening for negative pressure-tight fluidic connection of the wound space to a negative pressure source is formed in the cover layer. When a negative pressure wound dressing is applied as intended onto a wound and the negative pressure source is fluidically connected in a negative pressure-tight fashion to the attachment opening, a negative pressure can be established in the wound space by the negative pressure source. Typically, a connecting device that comprises an elongate single-lumen or multilumen connecting tube is used for the negative pressure-tight connection of the negative pressure wound dressing to the negative pressure source. A proximal end portion of the connecting tube can be fluidically connected in a negative pressure-tight fashion to the negative pressure source. A distal end portion of the connecting tube can be fluidically connected in a negative pressure-tight fashion to the negative pressure wound dressing. In order to prevent negative pressure-induced collapse of the connecting tube during use in negative pressure therapy, a supporting unit is typically arranged in a tube lumen of the connecting tube. The supporting unit is permeable for fluid, for example air and in particular wound exudate, in the longitudinal extent of the tube lumen.

A connecting device of the species is known, for example, from the published patent application WO 2012 087 376 A1. This proposes various embodiments of the supporting unit. For example, the supporting unit may comprise an open-celled foam, a spacer fabric or a nonwoven. The published patent application WO 2013 175 306 A1 describes a further connecting device of the species. According thereto, the supporting unit may comprise a porous material, for example a three-dimensional knitted fabric. Although the supporting units described in WO 2012 087 376 A1 and WO 2013 175 306 A1 fulfill the basic requirements of a supporting unit, the proposed materials lead however to a comparatively high cost burden. This has been tolerated in previously known connecting devices.

The object of the invention is to provide a way of ensuring effective support of the connecting tube with economical means in a connecting device for negative pressure wound therapy.

This object is achieved according to the invention by a connecting device as claimed in claim 1, by a negative pressure wound therapy kit as claimed in claim 21 and by a negative pressure therapy system as claimed in claim 22.

The dependent claims and the description specify advantageous variants and embodiments.

The invention thus provides a connecting device for the negative pressure-tight fluidic connection of a negative pressure wound dressing to a negative pressure source. The connecting device comprises an elongate single-lumen or multilumen connecting tube, in particular a film tube, which comprises a distal end portion and a proximal end portion. The distal end portion can be fluidically connected in a negative pressure-tight fashion to the negative pressure wound dressing. The proximal end portion can be fluidically connected in a negative pressure-tight fashion to the negative pressure source.

The terms “distal” and “proximal” describe the arrangement relative to the negative pressure source. A proximal portion of an element is closer to the negative pressure source than a distal portion of the same element is. In this respect, for example, the proximal end portion of the connecting tube is closer to the negative pressure source than the distal end portion is when the connecting tube is being used as intended.

A “negative pressure-tight fluidic connection” is intended here to mean that, taking into account the negative pressure source used, the negative pressure needed for negative pressure wound therapy can be sustained in the cavities that are fluidically connected to one another. Typically, in the scope of negative pressure therapy, a pressure difference between the air pressure inside the negative pressure wound dressing and the ambient air pressure which is from at least 20 mm Hg (millimeters of mercury column) to at most 250 mm Hg is set up. 1 mm Hg corresponds to one torr, i.e. 133.322 Pa (pascals).

The connecting device according to the invention moreover comprises a supporting unit, which is arranged in a tube lumen of the connecting tube. The supporting unit supports the connecting tube against collapse, in particular collapse induced by negative pressure, and is permeable for fluid, for example air and in particular wound exudate, in the longitudinal extent of the tube lumen.

Now, the supporting unit comprises at least one flat material web portion, the flat material web portion is configured in a structured fashion by elevations formed integrally with a plane of the flat material web portion in order to support the connecting tube, and a continuous intermediate space is formed between the elevations so that fluid, for example air and in particular wound exudate, can be transported through the intermediate space in the longitudinal extent of the tube lumen.

It has surprisingly been discovered that a flat material web portion configured as above is particularly suitable in respect of its properties as a supporting unit or as a component of a supporting unit. The continuous intermediate space is formed between the elevations according to the invention. Through the liquid-permeable intermediate space, a negative pressure can communicate by means of a negative pressure source into a negative pressure wound dressing. Owing to this negative pressure, a tube wall of the connecting tube experiences an inwardly directed pressure force. In particular, this pressure force leads to a wall portion of the tube wall, which lies opposite the elevations, clinging onto the raised end sides of the elevations. The continuous intermediate space is not thereby sealed, however, so that collapse of the connecting tube is reliably prevented. Collapse of the connecting tube means sealing of the connecting tube.

A flat material web portion configured according to the invention may be obtained economically. This is due, in particular, to the fact that the forming of the elevations may advantageously be integrated into the production of the flat material web portion. The use of a flat material web portion as a supporting unit, or as a component of a supporting unit, furthermore has the advantage that a connecting tube may be produced with a small overall height. Such a flat connecting tube is associated with a high degree of patient comfort.

A flat material web portion is a portion of a flat material web. For example, the flat material web portion is cut out from a flat material web. Preferably, the elevations are already formed in the flat material web. The elevations may, however, also be formed later in the flat material web portion. The flat material web is in particular a plastic film web, so that the flat material web portion is configured as a plastic film.

Preferably, the flat material web portion has an elevation density of at least 50 elevations per cm², preferably an elevation density of at least 100 elevations per cm², particularly preferentially an elevation density of at least 200 elevations per cm². Such high elevation densities are associated with small-dimensioned elevations. The stability of the flat material web portion is then impaired at most slightly by the elevations and end-side through-openings that are optionally present. On the other hand, it is also advantageous to limit the elevation density in order to achieve distances between neighboring elevations that are sufficient large for the fluid permeability. Preferably, the flat material web portion has an elevation density of at most 1000 elevations per cm², preferentially an elevation density of at most 500 elevations per cm². Particularly preferentially, the flat material web portion has an elevation density of from at least 200 elevations per cm² to at most 400 elevations per cm².

The connecting tube may be configured with a single lumen or multiple lumens. In the case of a single-lumen connecting tube, there is only one lumen, which extends from the distal end portion to the proximal end portion. When the connecting device is being used as intended, the tube lumen acts as a suction lumen. In the case of a multilumen connecting tube, the tube lumen is subdivided into a plurality of partial lumens, each of which extends from the distal end portion to the proximal end portion. The partial lumens are preferably connected in parallel with one another to an inlet opening formed in the distal end portion, each of the partial lumens being assigned its own passage in the region of the proximal end portion. In such a multilumen connecting tube, a first of the partial lumens acts as a suction lumen when the connecting device is being used as intended. A second of the partial lumens may, for example, be used as an irrigation lumen and/or as a measurement lumen.

According to one preferred embodiment, the elevations are configured conically, cylindrically, frustopyramidally or hyperboloidally. The tube wall of the connecting tube can be supported effectively by elevations shaped in such a way. They moreover provide a highly branched intermediate space. This has the advantage that the transport of fluid is possible even if the intermediate space is blocked at isolated points, for example between two neighboring elevations. Preferably, the elevations are arranged isolated from one another in an insular fashion. Preferably, the elevations have a height of from 200 μm to 1000 μm.

According to one preferred embodiment, at least in a central region of the flat material web portion, each of the elevations is surrounded by from three to eight further elevations, the further elevations together forming a regular polygon. Such an arrangement of the elevations ensures particularly effective support of the tube wall. Particularly preferentially, the further elevations form a regular hexagon.

According to one preferred embodiment, the elevations are formed on a first side of the flat material web portion and a second side of the flat material web portion, which faces away from the first side, is configured smoothly. It has been found that elevations on only one side of the flat material web portion are sufficient for effective support of the tube wall. In addition, a continuous intermediate space on only one side of the flat material web portion also provides sufficient permeability for fluid in the longitudinal extent of the tube lumen. A flat material web portion having a smooth second side is easy to produce in terms of manufacturing technology.

A “smooth side” of a layer is not intended here to mean that the smooth side is necessarily free of any unevenness. Rather, a “smooth side” indicates that it is smooth in comparison with the first side of the flat material web portion, which is structured by the elevations. Accordingly, there may also be unevenness on a smooth side of a layer, for example due to irregularities in the material thickness of the layer in question. In respect of its height, however, any unevenness belongs to a lower order of magnitude than the elevations of the structured first side of the flat material web portion.

Preferably, the elevations are formed by vacuum deep-drawing. Such a way of forming the elevations may be integrated expeditiously into the production of the flat material web, or of the flat material web portion. For example, a calendered or extruded, still molten flat material web, in particular a plastic film web, may for this purpose be guided over a rotating perforated negative pressure roll. The flat material web is then locally drawn into holes of the perforated negative pressure roll by a negative pressure in the perforated negative pressure roll, so that the elevations are formed. The shape of the elevations that are obtained is determined, for example, by the contour of the holes and by the level of the negative pressure used.

According to one preferred embodiment, the flat material web portion is made of polyethylene, polyurethane, polyvinyl chloride or a mixture thereof. These plastics are particularly suitable for forming the flat material web portion because of their properties. On the one hand, the plastics are sufficiently flexible, which is associated with a high degree of patient comfort. For example, pressure ulcers may be avoided by a flexible embodiment of the connecting tube. In addition, a sufficiently stiff flat material web portion may be produced, so that the elevations can support the tube wall reliably.

According to one preferred embodiment, an end-side through-opening is formed in at least some of the elevations, particularly in all of the elevations. The distribution of fluid, in particular wound exudate, in the tube lumen may be improved by the end-side through-openings. Specifically, through the end-side through-openings, fluid can pass through the flat material web portion and then be distributed on both sides of the flat material web portion in the tube lumen. The end-side through-openings may be formed simply in terms of manufacturing technology. Preferably, the negative pressure used during the vacuum deep-drawing is adjusted in such a way that the end sides of at least some of the elevations are open. The elevations and the end-side through-openings are then formed in the same method step. The presence of the end-side through-openings is preferred. Nevertheless, the disclosure also includes flat material web portions in which there are no end-side through-openings.

Preferably, the diameter of the end-side through-openings is at least 100 μm, preferentially at least 200 μm. Preferably, the diameter of the end-side through-openings is at most 1000 μm, preferentially at most 500 μm. Particularly preferentially, the diameter of the end-side through-openings is at least 200 μm and at most 500 μm.

According to one preferred embodiment, at least one planar-side through-opening is formed in the plane of the flat material web portion. The distribution of fluid, in particular wound exudate, in the tube lumen may also be improved by one or more planar-side through-openings. In addition, the planar-side through-openings have the advantage that they can be formed independently of the elevations. For example, the planar-side through-openings are formed in the flat material web, or in the flat material web portion, by stamping after formation of the elevations and any end-side through-openings. In particular, the planar-side through-openings are arranged between the elevations. The planar-side through-openings may, however, also be superimposed with the elevations. This is the case, for example, when the planar-side through-openings are formed by stamping, a part of an elevation also being stamped out during the stamping. Preferably, the planar-side through-openings are present in addition to the end-side through-openings. The planar-side through-openings may, however, also be present instead of the end-side through-openings. Moreover, the disclosure also includes flat material web portions in which there are neither planar-side through-openings nor end-side through-openings.

Preferably, the flat material web portion extends into the distal end portion. Accordingly, the distal end portion is also supported against collapse by the flat material web portion.

According to one preferred embodiment, at least one planar-side through-opening is formed in the plane of the flat material web portion in the region of the distal end portion. Such an arrangement is particularly preferred for planar-side through-openings. When the connecting device is being used in negative pressure therapy, wound exudate enters the connecting tube in the region of the distal end portion. By the planar-side through-openings in the region of the distal end portion, the permeability of the flat material web portion is increased in this region. Accordingly, wound exudate can already be distributed effectively on both sides of the flat material web portion in the region of the distal end portion. In particular, there are one or more planar-side through-openings only in the region of the distal end portion.

According to one preferred embodiment, the connecting tube comprises an elongate middle portion and the distal end portion is two-dimensionally widened in relation to the middle portion. The widening of the distal end portion increases the contact area between the distal end portion and the negative pressure wound dressing. A connection between the negative pressure wound dressing and the distal end portion that is particularly robust mechanically may be achieved by the increase of the contact area. Moreover, any unevenness in the negative pressure wound dressing may be compensated for more easily by the two-dimensionally widened distal end portion. Preferably, the distal end portion is widened in the shape of a circular disk or rectangularly.

According to one preferred embodiment, the connecting tube comprises a first ply and a second ply, the plies being connected to one another in their edge regions and together enclosing the tube lumen. Such a multiply embodiment of the connecting tube facilitates the arrangement of the supporting unit in the tube lumen. In the scope of the production of the connecting device, for example, the first ply, the supporting unit and the second ply are stacked above one another. Only then are the first ply and the second ply connected to one another in their edge regions.

According to one preferred embodiment, the first ply is configured at least substantially in a planar fashion and the flat material web portion extends parallel to the first ply. A planar first ply has the advantage that two-dimensional application of the connecting tube on the wound dressing, or on surrounding tissue, can be ensured. Alignment of the flat material web portion parallel to the first ply is preferred because this prevents the flat material web portion from being creased or otherwise deformed when the connecting device is being used in negative pressure therapy.

According to one preferred embodiment, the distal end portion comprises at least one inlet opening and the inlet opening is formed in the planar first ply. This facilitates the negative pressure-tight connection of the distal end portion to the negative pressure wound dressing. The planar first ply may advantageously be matched to a region of the cover layer of the negative pressure wound dressing that comprises the attachment opening.

According to one preferred embodiment, the elevations extend away from the first ply. In the elevations, the wound exudate is transported by capillary forces in the direction of the end-side through-openings. If the elevations extend away from the first ply, the transport of wound exudate through the flat material web portion is accordingly reinforced by the capillary forces. The wound exudate may then be distributed effectively on both sides of the flat material web portion.

According to one preferred embodiment, the flat material web portion is inserted loosely into the tube lumen. Such a connecting device may be produced expeditiously and with few working steps in the scope of production. Alternatively, the flat material web portion is connected to the connecting tube, for example by an adhesive bond or by a welded connection.

According to one preferred embodiment, a resiliently deformable attachment plate is arranged at the distal end portion, the tube lumen being fluidically connected to the surroundings through at least one through-opening formed in the attachment plate. The tube lumen can then be fluidically connected in a negative pressure-tight fashion to the negative pressure wound dressing through the through-opening in the attachment plate. The contact area between the connecting device and the negative pressure wound dressing is increased by the attachment plate, so that a secure connection can be established. Preferably, a first side of the attachment plate, which faces away from the distal end portion, is configured to be adhesive at least in portions. For example, a double-sided adhesive tape may be arranged on the first side of the attachment plate.

According to one preferred embodiment, the supporting unit comprises at least one further flat material web portion, the further flat material web portion is configured in a structured fashion by elevations formed integrally with a plane of the further flat material web portion in order to support the connecting tube, a continuous intermediate space is formed between the elevations so that fluid, for example air and in particular wound exudate, can be transported through the intermediate space in the longitudinal extent of the tube lumen, and the flat material web portion and the further flat material web portion are arranged stacked above one another. By providing at least one further flat material web portion, a tube lumen having a larger cross section can be supported.

Regarding the further configuration of the further flat material web portion, the features described in connection with the flat material web portion may be employed. Preferably, the flat material web portions are configured identically. The flat material web portions may, however, also differ from one another in at least one feature, for example in the shape of the elevations and/or the material composition. The presence of a plurality of flat material web portions arranged stacked above one another is preferred. Nevertheless, the disclosure also includes supporting units that have only one flat material web portion.

Preferably, the flat material web portions are arranged in such a way that the elevations of the flat material web portions protrude in the same direction. This offers the advantage that the intermediate space between the elevations of the one flat material web portion are not blocked by the elevations of the further flat material web portion. Such a blocking of the intermediate space could otherwise impair the transport of fluid.

According to one preferred embodiment, an air-permeable and liquid-impermeable filter unit, which has a filtering effect between an inlet of the connecting tube and an outlet of the connecting tube, is arranged in the tube lumen. Because the filter unit is configured to be air-permeable, a negative pressure can communicate through the filter unit into the reception space. Because the filter unit is configured to be liquid-impermeable, the filter unit prevents liquid, for example wound exudate, from reaching the downstream negative pressure source when the connecting device is being used in negative pressure therapy. The negative pressure source could otherwise be contaminated and/or damaged. Preferably, the filter unit is arranged in the region of the proximal end portion.

The object to be achieved is also achieved by a negative pressure wound therapy kit which comprises a negative pressure wound dressing and a connecting device having the features described above, wherein the distal end portion of the connecting device can be fluidically connected in a negative pressure-tight fashion to the negative pressure wound dressing, and wherein the proximal end portion of the connecting device can be fluidically connected in a negative pressure-tight fashion to a negative pressure source.

In respect of the advantages achievable with the negative pressure wound therapy kit, reference is made to the relevant comments relating to the connecting device. Regarding the further configuration of the negative pressure wound therapy kit, the features described in connection with the connecting device may be employed.

The object to be achieved is also achieved by a negative pressure wound therapy system which comprises a negative pressure wound therapy kit having the features described above. The negative pressure wound therapy system according to the invention moreover comprises a negative pressure source, wherein the proximal end portion of the connecting device can be fluidically connected in a negative pressure-tight fashion to the negative pressure source.

In respect of the advantages achievable with the negative pressure wound therapy system, reference is made to the relevant comments relating to the connecting device. Regarding the further configuration of the negative pressure wound therapy system, the features described in connection with the connecting device may be employed.

The invention is described in more detail below with the aid of the figures, identical or functionally equivalent elements sometimes being provided with reference signs only once. The figures serve as an example and are not to be interpreted as restrictive. In the figures:

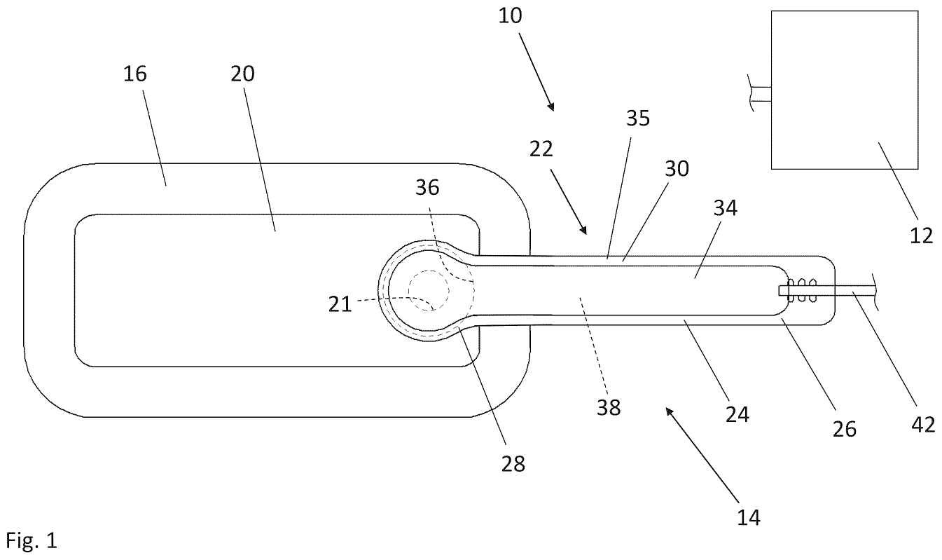

FIG. 1 shows a negative pressure wound therapy system having a connecting device, which comprises an elongate connecting tube,

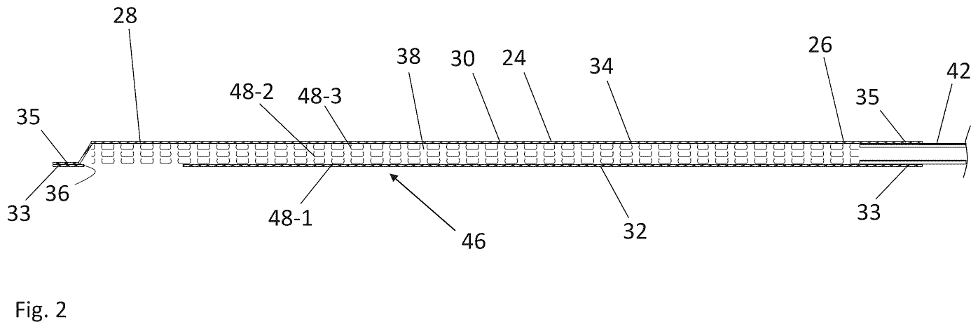

FIG. 2 shows a longitudinal section through the connecting tube represented in FIG. 1,

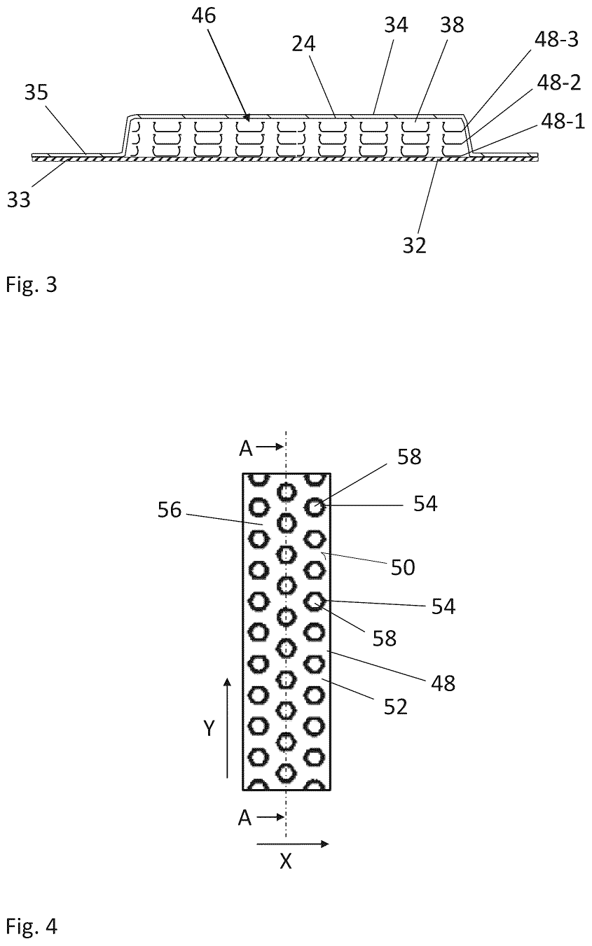

FIG. 3 shows a cross section through the connecting tube represented in FIG. 1,

FIG. 4 shows a plan view of a flat material web portion which is arranged in a tube lumen of the connecting tube,

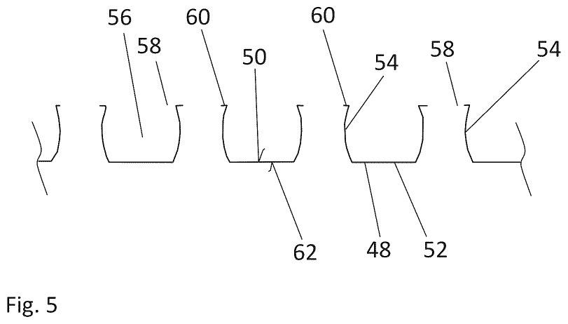

FIG. 5 shows a sectional representation along the section line A-A of the flat material web portion represented in FIG. 4,

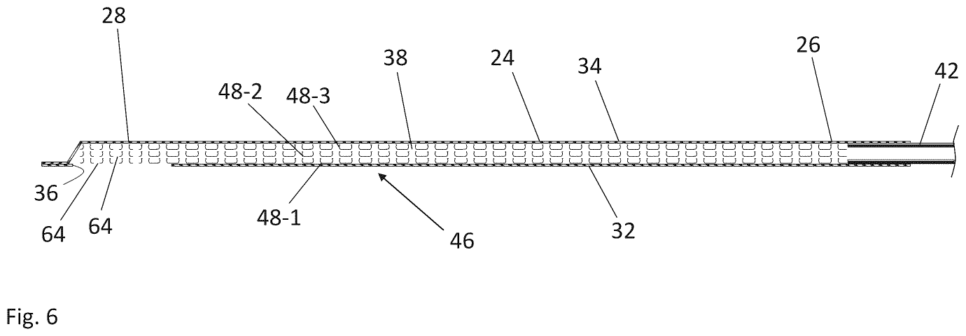

FIG. 6 shows a longitudinal section through a further exemplary embodiment of the connecting tube,

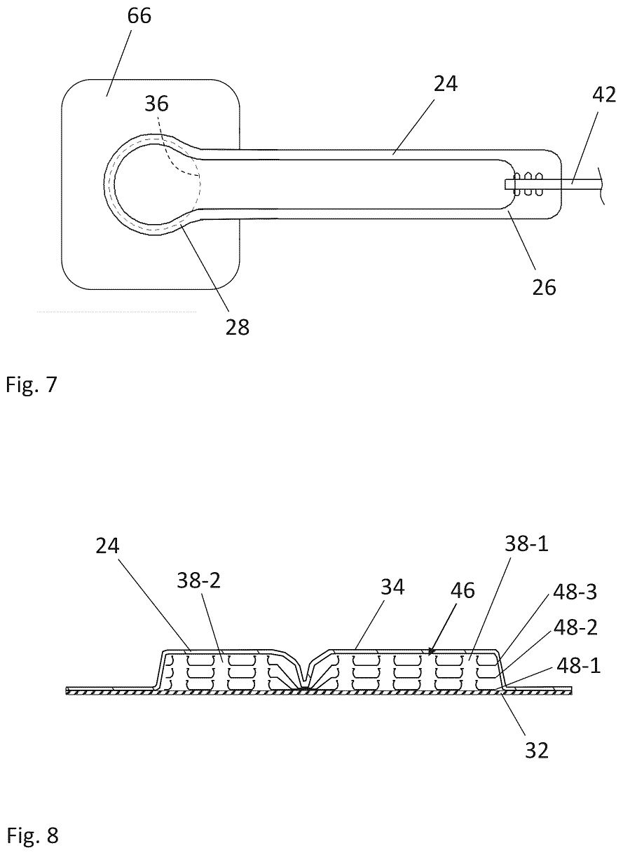

FIG. 7 shows a plan view of a further exemplary embodiment of the connecting device, and

FIG. 8 shows a cross section through a further exemplary embodiment of the connecting tube.

FIG. 1 shows a negative pressure wound therapy system 10 for use in negative pressure wound therapy. The negative pressure wound therapy system 10 comprises a negative pressure source 12 and a negative pressure wound therapy kit 14. The negative pressure wound therapy kit 14 comprises a negative pressure wound dressing 16, which is referred to below as the wound dressing 16. The wound dressing 16 comprises a wound contact layer, which is to be applied onto a wound bed of the wound, and an air-impermeable cover layer 20. In FIG. 1, the wound contact layer is covered by the cover layer 20 and therefore cannot be seen. The negative pressure wound therapy kit 14 moreover comprises a connecting device 22. The connecting device 22 comprises an elongate connecting tube 24 having a proximal end portion 26 and a distal end portion 28. The proximal end portion 26 can be fluidically connected in a negative pressure-tight fashion to the negative pressure source 12. The distal end portion 28 can be fluidically connected in a negative pressure-tight fashion to an attachment opening 21 formed in the cover layer 20. In the case of the negative pressure wound therapy system 10 represented in FIG. 1, the distal end portion 28 is already connected to the attachment opening 21 so that the attachment opening 21 is covered by the distal end portion 28.

The distal end portion 28 is two-dimensionally widened in relation to an elongate middle portion 30 of the connecting tube 24. In the present case, the distal end portion 28 is widened in the shape of a circular disk. The widening of the distal end portion 28 facilitates the connection of the distal end portion 28 to the attachment opening 21 of the wound dressing 16.

The construction of the connecting device 22 is explained in more detail below with additional reference to FIGS. 2 and 3. In this regard, FIGS. 2 and 3 respectively show a schematic longitudinal section and a schematic cross section through the connecting tube 24 represented in FIG. 1.

The connecting tube 24 in the present exemplary embodiment comprises a lower, or first ply 32 and an upper, or second ply 34. The plies 32 and 34 are connected to one another in their edge regions 33 and 35 and together enclose a tube lumen 38 of the connecting tube 24. The first ply 32 and the second ply 34 form the tube wall of the connecting tube 24. In the present case, the plies 32 and 34 are formed as film plies. Accordingly, the connecting tube 24 is a film tube.

Preferably, the plies 32 and 34 are made of polyurethane, polyvinyl chloride, polyethylene, silicone or a mixture thereof. Preferably, the edge regions 33 and 35 of the plies 32 and 34 are connected by an adhesive bond or by a welded connection. An adhesive bond may, for example, be formed by applying an adhesive or by means of an adhesive tape. A welded connection may, for example, be formed by heat or by ultrasound.

The first ply 32 is configured at least substantially in a planar fashion. If the connecting device 22 is connected to the wound dressing 16 as intended, the first ply 32 faces toward the wound dressing 16. Because the first ply 32 is configured at least substantially in a planar fashion, two-dimensional application of the first ply 32 on the wound dressing 16, or on surrounding tissue, is ensured. The second ply 34 is configured to be outwardly convex.

The distal end portion 28 comprises an inlet opening 36. The inlet opening 36 is formed in the first ply 32. If the distal end portion 28 is connected as intended to the wound dressing 16 as shown in FIG. 1, the attachment opening 21 in the wound dressing 16 and the inlet opening 36 are brought to overlap at least partially with one another.

In the exemplary embodiment represented in FIGS. 1, 2 and 3, the connecting tube 24 is configured with a single lumen, as may be seen in FIG. 3. Accordingly, the tube lumen 38 is not subdivided into a plurality of partial lumens. When the connecting device 22 is being used as intended, the tube lumen 38 acts as a suction lumen. In order to connect the tube lumen 38 to the negative pressure source 12, an attachment element 42 protrudes into the tube lumen 38 in the region of the proximal end portion 26 through a passage formed between the edge region 33 of the first ply 32 and the edge region 35 of the second ply 34.

The connecting device 22 moreover comprises a supporting unit 46, which supports the connecting tube 24 against collapse, in particular collapse induced by negative pressure. The supporting unit 46 comprises a plurality of flat material web portions 48-1, 48-2 and 48-3. The flat material web portions 48-1, 48-2 and 48-3 are arranged in the tube lumen 38 and extend in the longitudinal extent of the connecting tube 24. In the present case, the flat material web portions 48-1, 48-2 and 48-3 extend from the proximal end portion 26 into the distal end portion 28.

The configuration of the flat material web portions 48 is explained in more detail below with additional reference to FIGS. 4 and 5. FIG. 4 in this regard shows a plan view of one of the flat material web portions 48. It should be pointed out that FIG. 4 only represents a detail of the flat material web portion 48. Accordingly, the actual outer contour of the flat material web portion 48 differs from the outer contour shown in FIG. 4. FIG. 5 shows a sectional representation of the flat material web portion 48 along the section line A-A shown in FIG. 4.

The flat material web portion 48 is elongated in two surface directions X and Y. In order to support the connecting tube 24, a first side 50 of the flat material web portion 48 is configured in a structured fashion by elevations 54 formed integrally with a plane 52 of the flat material web portion 48. A continuous intermediate space 56 is formed between the elevations 54. Fluid, for example air and in particular wound exudate, can be transported through the intermediate space 56 in the longitudinal extent of the tube lumen 38.

In at least some of the elevations 54, an end-side through-opening 58 is formed in the raised end sides 60 of the elevations 54 in question. In the present case, an end-side through-opening 58 is respectively formed in all of the elevations 54. Through the end-side through-openings 58, fluid can pass through the flat material web portion 48 and then be distributed on both sides of the flat material web portion 48. The second side 62 of the flat material web portion 48, which faces away from the structured first side 50, is configured smoothly.

Preferably, the flat material web portion 48 has an elevation density of from at least 200 elevations 54 per cm² to at most 400 elevations 54 per cm² on the first side 50. In the present case, the elevation density is about 280 elevations 54 per cm². Preferably, the diameter of the end-side through-openings 58 is at least 200 μm and at most 500 μm. The height of the elevations 54 is preferably at least 200 μm and at most 1000 μm.

In order to form the continuous intermediate space 56, the elevations 54 are arranged isolated from one another in an insular fashion. In the exemplary embodiment represented, the elevations 54 are equivalent in their shape to a one-sheeted hyperboloid. In the present case, the elevations 54 are thus configured hyperboloidally. A lateral wall of the elevations 54 is for this purpose curved in an anticlastic fashion. Alternatively, the elevations 54 are preferably configured conically, i.e. frustoconically, cylindrically or frustopyramidally.

The elevations 54 are arranged distributed in such a way that, at least in a central region of the flat material web portion 48, each of the elevations 54 is surrounded by from three to eight further elevations 54, the further elevations 54 together forming a regular polygon. In the present case, the further elevations 54 together form a regular hexagon. At least in the central region of the flat material web portion 48, each of the elevations 54 is thus surrounded by six further elevations 54.

The flat material web portion 48 is in the present case a plastic film 48. The plastic film 48 is preferably made of polyethylene, polyurethane, polyvinyl chloride or a mixture thereof. These plastics have the advantage that a flexible connecting tube 24 may be obtained, which is associated with a high degree of patient comfort. For example, the occurrence of pressure ulcers may be avoided by the flexible embodiment of the connecting tube 24. In addition, the aforementioned plastics also have a sufficient stiffness. In this respect, the plastics ensure that the continuous intermediate space 56 is preserved when the connecting device 22 is being used, despite the negative pressure that is established.

The elevations 54 are made by vacuum deep-drawing. Preferably, for this purpose an extruded or calendered, still meltable flat material web, in particular a plastic film web, is guided over a rotating perforated negative pressure roll. The flat material web is then locally drawn into holes of the perforated negative pressure roll by a negative pressure in the perforated negative pressure roll, so that the elevations 54 are formed. The shape of the elevations 54 that are obtained is determined, for example, by the contour of the holes and by the negative pressure used. Preferably, the negative pressure in the perforated negative pressure roll is adjusted in such a way that the end sides 60 of the elevations 54 are open. The elevations 54 and the end-side through-openings 58 are thus formed in the same method step.

The flat material web portions 48 are arranged parallel to the planar first ply 32 in the tube lumen 38. The flat material web portions 48 are arranged in such a way that the elevations 54 extend in the direction of the second ply 34, i.e. away from the first ply 32. This reinforces the transport of wound exudate through the flat material web portions 48 in the direction of the second ply 34, so that the wound exudate is distributed on both sides of the flat material web portions 48. Specifically, capillary forces act on the wound exudate in the elevations 54 and promote the transport in the direction of the second ply 34.

In the exemplary embodiment represented in FIGS. 1, 2 and 3, three flat material web portions 48 are arranged in the tube lumen 38. There may, however, also be a number of flat material web portions 48 other than three. Accordingly, there may also be more than three flat material web portions 48 or fewer than three flat material web portions 48.

The flat material web portions 48 are inserted loosely into the tube lumen 38. As an alternative thereto, the flat material web portions 48 are connected to the first ply 32 and/or to the second ply 34, for example by an adhesive bond or by a welded connection.

FIG. 6 shows a representation, which corresponds to FIG. 2, of a further exemplary embodiment of the connecting tube 24. The connecting tube 24 represented in FIG. 6 differs from the connecting tube 24 represented in FIG. 2 in that a plurality of planar-side through-openings 64 are formed in the plane 52 of the flat material web portion 48 in the region of the distal end portion 28. For example, these planar-side through-openings 64 are formed by stamping in the flat material web portions 48. The planar-side through-openings 64 may already be formed in the flat material web or formed later in the flat material web portions 48. The planar-side through-openings 64 increase the permeability of the flat material web portions 48 for wound exudate. The presence of planar-side through-openings 64 is advantageous especially in the region of the distal end portion 28 because effective distribution of wound exudate orthogonally with respect to the surface directions X and Y already takes place in the distal end portion 28.

In the exemplary embodiment represented in FIG. 6, the planar-side through-openings 64 are present in addition to the end-side through-openings 58. According to a further exemplary embodiment, the planar-side through-openings 64 are present instead of the end-side through-openings 58. In such an exemplary embodiment, one or more planar-side through-openings 64 are preferably also formed in the flat material web portions 48 outside the distal end portion 28.

FIG. 7 shows a further exemplary embodiment of the connecting device 22. In the connecting device 22 represented in FIG. 7, a resiliently deformable attachment plate 66 is arranged at the distal end portion 28. The tube lumen 38 is fluidically connected to the surroundings through at least one through-opening formed in the attachment plate 66, said through-opening not being visible in FIG. 7. The through-opening in the attachment plate 66 is brought to overlap at least partially with the inlet opening 36 of the distal end portion 28. A connection between the wound dressing 16 and the connecting device 22 that is particularly robust mechanically may be established by the attachment plate 66. Specifically, the contact area between the connecting device 22 and the cover layer 20 of the wound dressing 16 is increased by the attachment plate 66. Preferably, a first side of the attachment plate 66, which faces away from the distal end portion 28, is configured to be adhesive at least in portions. Accordingly, an adhesive bond may be established between the attachment plate 66 and the cover layer 20.

FIG. 8 shows a representation, which corresponds to FIG. 3, of a further exemplary embodiment of the connecting tube 24. In the exemplary embodiment represented in FIG. 8, the connecting tube 24 is configured with multiple lumens. The tube lumen 38 is for this purpose subdivided into a first partial lumen 38-1 and a second partial lumen 38-2.

The two partial lumens 38-1 and 38-2 are connected in parallel with one another to the inlet opening 36 of the distal end portion 28. In the region of the proximal end portion 26, the two partial lumens 38-1 and 38-2 each comprise their own passage. When the multilumen connecting tube 24 is being used as intended, the first tube lumen 38-1 is used as a suction lumen. The second tube lumen 38-2 may, for example, be used as an irrigation lumen and/or as a measurement lumen.

In order to form the two partial lumens 38-1 and 38-2, the first ply 32 is locally connected materially, preferably welded, to the second ply 34.

In the exemplary embodiment represented, the flat material web portions 48 extend both in the first partial lumen 38-1 and in the second partial lumen 38-2. The material connection between the first ply 32 and the second ply 34 is an indirect material connection, namely a material connection by means of the support layers 48 of the supporting unit 46. Preferably, the indirect materially fluid connection is established by welding. The first ply 32, the second ply 34 and the support layers 48 lying between them are in this case locally heated to a temperature above the melting temperature and thereby welded together. Specifically, the first ply 32 is in the present case directly connected materially to the first flat material web portion 48-1. The second ply 34 is directly connected materially to the third flat material web portion 48-3. In addition, the flat material web portions 48 are materially connected to one another.

According to a further exemplary embodiment, the flat material web portions 48 extend only in the first partial lumen 38-1, which is used as a suction lumen. With such an arrangement of the flat material web portions 48, the material connection between the first ply 32 and the second ply 34 is preferably a direct material connection.

1-22. (canceled)

23. A connecting device for negative pressure-tight fluidic connection of a negative pressure wound dressing to a negative pressure source, said device comprising:
an elongate single-lumen or multilumen connecting tube, which comprises a distal end portion and a proximal end portion, wherein the distal end portion is able to be fluidically connected in a negative pressure-tight fashion to the negative pressure wound dressing, and wherein the proximal end portion is able to be fluidically connected in a negative pressure-tight fashion to the negative pressure source; and
a supporting unit, which is arranged in a tube lumen of the connecting tube, wherein the supporting unit supports the connecting tube against collapse and is permeable for fluid in the longitudinal extent of the tube lumen, wherein the supporting unit comprises at least one flat material web portion,
wherein the flat material web portion is configured in a structured fashion by elevations formed integrally with a plane of the flat material web portion in order to support the connecting tube, and
wherein a continuous intermediate space is formed between the elevations so that fluid is able to be transported through the intermediate space in the longitudinal extent of the tube lumen.

24. The connecting device of claim 23, wherein the elevations are configured conically, cylindrically, frustopyramidally or hyperboloidally.

25. The connecting device of claim 23, wherein at least in a central region of the flat material web portion, each of the elevations is surrounded by from three to eight further elevations, the further elevations together forming a regular polygon.

26. The connecting device of claim 23, wherein the elevations are formed on a first side of the flat material web portion, and wherein a second side of the flat material web portion, which faces away from the first side, is configured smoothly.

27. The connecting device of claim 23, wherein an end-side through-opening is formed in at least some of the elevations.

28. The connecting device of claim 23, wherein at least one planar-side through-opening is formed in the plane of the flat material web portion.

29. The connecting device of claim 23, wherein the flat material web portion extends into the distal end portion.

30. The connecting device of claim 29, wherein at least one planar-side through-opening is formed in the plane of the flat material web portion in the region of the distal end portion.

31. The connecting device of claim 23, wherein the connecting tube comprises an elongate middle portion, and wherein the distal end portion is two-dimensionally widened in relation to the middle portion.

32. The connecting device of claim 23, wherein the connecting tube comprises a first ply and a second ply, wherein the first ply and second ply each comprise an edge region, and wherein the first ply and second ply are connected to one another in the edge regions and together enclose the tube lumen.

33. The connecting device of claim 32, wherein the first ply is configured at least substantially in a planar fashion, and wherein the flat material web portion extends parallel to the first ply.

34. The connecting device of claim 33, wherein the distal end portion comprises at least one inlet opening, and wherein the inlet opening is formed in the first ply.

35. The connecting device of claim 34, wherein the elevations of the flat material web portion extend away from the first ply.

36. The connecting device of claim 23, wherein the flat material web portion is inserted loosely into the tube lumen, or wherein the flat material web portion is connected to the connecting tube.

37. The connecting device of claim 23, wherein a resiliently deformable attachment plate is arranged at the distal end portion, the tube lumen being fluidically connected to the surroundings through at least one through-opening formed in the attachment plate.

38. The connecting device of claim 23, wherein the supporting unit comprises at least one further flat material web portion, wherein the further flat material web portion is configured in a structured fashion by elevations formed integrally with a plane of the further flat material web portion in order to support the connecting tube, wherein a continuous intermediate space is formed between the elevations so that fluid is able to be transported through the intermediate space in the longitudinal extent of the tube lumen, and wherein the flat material web portion and the further flat material web portion are arranged stacked above one another.

39. The connecting device of claim 38, wherein the flat material web portions are arranged in such a way that the elevations of the flat material web portions protrude in the same direction.

40. The connecting device of claim 23, wherein an air-permeable and liquid-impermeable filter unit, which has a filtering effect between an inlet of the connecting tube and an outlet of the connecting tube, is arranged in the tube lumen.

41. A negative pressure wound therapy kit comprising a negative pressure wound dressing and comprising a connecting device as claimed in claim 23, wherein the distal end portion of the connecting device is able to be fluidically connected in a negative pressure-tight fashion to the negative pressure wound dressing, and wherein the proximal end portion of the connecting device is able to be fluidically connected in a negative pressure-tight fashion to a negative pressure source.

42. A negative pressure wound therapy system comprising a negative pressure wound therapy kit as claimed in claim 41 and comprising a negative pressure source, wherein the proximal end portion of the connecting device is able to be fluidically connected in a negative pressure-tight fashion to the negative pressure source.
